(19) United States
(12) Patent Application Publication
(10) Pub. No.: US 2010/0267959 A1
(43) Pub. Date: Oct. 21, 2010

(54) PROCESS FOR THE PREPARATION OF ESOMEPRAZOLE MAGNESIUM DIHYDRATE

(75) Inventors: Joseph Prabahar KOILPILLAI, Navi Mumbai Maharashtra (IN); Pravin Bhalchandra Kulkami, Maharashtra (IN); Avikumar Digambar Dabe, Maharastra (IN); Mubeen Ahmed Khan, Navi Mumbai Maharashtra (IN)

Correspondence Address: GLENMARK GENERICS INC., 750 CORPORATE DRIVE, MAHWAH, NJ, 07430, US

(73) Assignee: Glenmark Generics Limited, Mumbai (IN)

(21) Appl. No.: 12/499,278

(22) Filed: Jul. 8, 2009

(30) Foreign Application Priority Data
Apr. 15, 2009 (IN) 983/MUM/2009

(51) Int. Cl. C07D 401/12

(52) U.S. Cl. 546/273.7

(57) ABSTRACT

The present invention relates to a process for the preparation of esomeprazole magnesium dihydrate, specifically, esomeprazole magnesium crystalline dihydrate form A; and pharmaceutical compositions thereof.

### PRIORITY

This application claims the benefit to Indian Provisional Application 983/MUM/2009, filed on Apr. 15, 2009, the contents which is incorporated by reference herein.

### BACKGROUND OF THE INVENTION

1. Technical Field

The present invention relates to a process for the preparation of esomeprazole magnesium dihydrate, specifically, esomeprazole magnesium crystalline dihydrate form A; and pharmaceutical compositions thereof.

2. Description of the Related Art

Omeprazole is chemically known as 5-methoxy-2-[[(4-methoxy-3,5-dimethyl-2-pyridinyl) methyl]sulfinyl]-1H-benzimidazole. The S-enantiomer is chemically known as (S)-5-methoxy-2-[[(4-methoxy-3,5-dimethyl-2-pyridinyl)-methyl]sulfinyl]-1H-benzimidazole and hence named “esomeprazole”. Esomeprazole is a proton pump inhibitor used in the treatment of dyspepsia, peptic ulcer disease, gastroesophageal reflux disease and Zollinger-Ellison syndrome. Esomeprazole is the S-enantiomer of omeprazole (marketed as LOSEC®/PRILOSEC®).

The magnesium salt of esomeprazole in the form of trihydrate is marketed under the brand name NEXIUM® and is represented by formula I.

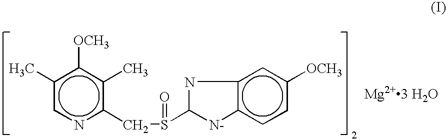

U.S. Pat. No. 5,714,505 describes alkaline salts of the (−) enantiomer of 5-methoxy-2-[[(4-methoxy-3,5-dimethyl-2-pyridiniyl)methyl]sulfinyl]-1H-benzimidazoles (i.e., esomeprazole) including the magnesium salt.

U.S. Pat. No. 6,369,085 (the '085 patent) describes crystalline esomeprazole magnesium trihydrate, the crystalline dihydrate forms A, and B and processes for their preparation. The '085 patent discloses the preparation of crystalline forms A and B using methanol, acetone and water as solvents.

The process for the preparation of esomeprazole magnesium dihydrate form A, using methanol, acetone and water, is not industrially efficient. The process results in the dihydrate being converted to the trihydrate or to the amorphous esomeprazole magnesium salt. This process is disadvantageous since the form A of the dihydrate as a wet dihydrate gets converted during drying to the trihydrate or amorphous form, leading to inconsistent production. Further, the use of acetone to slurry the product with subsequent drying, results in an inconsistent polymorph.

International patent publication WO2008102145 describes a process for the preparation of crystalline dihydrate form A of esomeprazole magnesium comprising crystallising or recrystallising crude (S)-5-methoxy-2-[[(4-methoxy-3,5-dimethyl-2-pyridinyl)-methyl]sulfinyl]-1H-benzimidazole magnesium dihydrate in the presence of ethyl acetate.

### SUMMARY OF THE INVENTION

The present invention relates to a process for the preparation of esomeprazole magnesium dihydrate; more specifically, esomeprazole magnesium crystalline dihydrate form A.

In one aspect, the present invention provides a process for the preparation of esomeprazole magnesium dihydrate comprising:
a) providing a suspension of esomeprazole magnesium by contacting it with one or more solvents or aqueous mixtures thereof; and,
b) precipitating the solid by stifling at about 30° C.; and
c) recovering the solid to obtain the crystalline dihydrate form of esomeprazole magnesium.

In yet another aspect, the present invention provides a process for the preparation of esomeprazole magnesium comprising:
a) contacting esomeprazole or a salt thereof with a magnesium source in the presence of a solvent or a mixture of solvents; and
b) separating the solids by filtration; and
c) concentrating the filtrate to obtain the crude esomeprazole magnesium.

In another aspect, the present invention provides esomeprazole magnesium dihydrate form A obtained by process herein described, with an X-ray diffractogram, which is substantially in accordance with FIG. 1.

In yet another aspect, the present invention provides esomeprazole magnesium dihydrate form A obtained by process herein described with a differential scanning calorimetry (DSC) endotherm curve, which is substantially in accordance with FIG. 2.

In a still further aspect, the present invention provides esomeprazole magnesium dihydrate form A obtained by process herein described with a thermogravimetric analysis (TGA) curve, which is substantially in accordance with FIG. 3.

In yet another embodiment, the present invention provides esomeprazole magnesium dihydrate form A having a purity at least about 99.8% as determined by chiral HPLC.

In a still further embodiment, the present invention provides esomeprazole magnesium dihydrate form A having a purity at least about 99.9% as determined by chiral HPLC.

In another embodiment, the present invention provides esomeprazole magnesium dihydrate form A having less than about 0.15% area of (R)-isomer impurity as determined by chiral HPLC.

In yet another embodiment, the present invention provides esomeprazole magnesium dihydrate form A having less than about 0.1% area of (R)-isomer impurity as determined by chiral HPLC.

In a still further embodiment, the present invention provides esomeprazole magnesium dihydrate form A having less than about 0.05% area of (R)-isomer impurity as determined by chiral HPLC.

In yet another embodiment, Esomeprazole magnesium dihydrate form A obtained by the process described herein has a residual organic solvent content of less than the amount recommended for pharmaceutical products, as set forth for example in ICH guidelines and U.S. pharmacopoeia; i.e., less than about 2000 ppm of Isopropyl alcohol, less than about 200 ppm of methanol and dichloromethane, less than about 5000 ppm of methyl tertiary butyl ether, dimethyl sulfoxide in the range of about 200 ppm to about 600 ppm and acetic acid and toluene at below the detection limit.

In yet another aspect, the present invention relates to pharmaceutical composition comprising esomeprazole magnesium dihydrate Form A obtained by the process of present invention and at least one pharmaceutically acceptable carrier.

### BRIEF DESCRIPTION OF THE DRAWINGS

FIG. 1: is an X-ray powder diffractogram of form A of esomeprazole magnesium dihydrate prepared by Example 2.

FIG. 2: is a Differential scanning calorimetry thermogram curve of esomeprazole magnesium dihydrate form A prepared by Example 2.

FIG. 3: is a thermogravimetric analysis (TGA) curve of esomeprazole magnesium dihydrate form A prepared by Example 2.

FIG. 4: is an X-ray powder diffractogram of form A of esomeprazole magnesium dihydrate after ball milling prepared by Example 3.

### DETAILED DESCRIPTION OF THE INVENTION

The present invention relates to a process for the preparation of esomeprazole magnesium dihydrate; more specifically, esomeprazole magnesium crystalline dihydrate form A.

As previously discussed, international patent publication WO2008102145 describes a process for the preparation of crystalline dihydrate form A of esomeprazole magnesium comprising crystallising or recrystallising crude (S)-5-methoxy-2-[[(4-methoxy-3,5-dimethyl-2-pyridinyl)-methyl]sulfinyl]-1H-benzimidazole magnesium dihydrate in the presence of ethyl acetate.

The use of ethyl acetate, which is water immiscible, poses a challenge. Ethyl acetate may solubilize the esomeprazole or its salts with a subsequent decrease in yield. Further, the use thereof requires additional process steps for recovery of solvent, which may render the process less cost-effective, thus unsuitable on a commercial scale.

The present invention provides an industrially suitable process for the preparation of esomeprazole magnesium dihydrate. More particularly, form A of esomeprazole magnesium crystalline dihydrate.

In one embodiment, the present invention provides a process for the preparation of esomeprazole magnesium dihydrate comprising:
a) providing a suspension of esomeprazole magnesium by contacting it with one or more solvents selected from the group alcohols, ethers or aqueous mixtures thereof; and
b) precipitating the solid by stirring at about 30° C.; and
c) recovering the solid to obtain the crystalline dihydrate form of esomeprazole magnesium.

The starting esomeprazole magnesium may be prepared by any of the known methods. Illustratively, as those in U.S. Pat. Nos. 5,714,504, 6,124,464; and 6,369,085, which are disclosed herein as references, in their entirety.

The solvents that can be used in a) of the process described above, for the preparation of suspension of esomeprazole magnesium is selected from methanol, ethanol, isopropyl alcohol, tertiary butyl alcohol, diethyl ether, methyl tertiary butyl ether and mixtures thereof, and their aqueous mixtures thereof in various proportions without limitation. Preferably, isopropyl alcohol and methyl tertiary butyl ether in combination with water.

The ratio of solvents methyl tertiary butyl ether, isopropyl alcohol and water is from about 0.2:0.2:0.05 to about 5:5:1 preferably the ratio is 0.45:0.45:0.1.

The temperature for the preparation of suspension in a) of the process can range from about 25° C. to about 35° C., preferably from about 25° C. to about 30° C.

The time period for the preparation of suspension can range from about 30 minutes to about 5 hours, preferably from about 30 minutes to 1 hour.

The precipitation of solid in b) above may be achieved by, but not limited to, evaporation, cooling, drying and the like. Preferably, by stifling from about 25° C. to about 30° C.

The temperature range for precipitation of solid can be from about −10° C. to about 30° C. Preferably from about 25° C. to about 30° C.

The time period for complete precipitation of solid can range from about 30 minutes to about 5 hours, preferably from about 1 hour to 4 hours.

Recovering the solid in c) to obtain the crystalline dihydrate form A of esomeprazole magnesium can be achieved by a conventional technique known in the art. Preferably, filtration.

In an embodiment, the filtration of solid esomeprazole magnesium dihydrate Form A has to be carried under nitrogen atmosphere. The obtained esomeprazole magnesium dihydrate form A can be dried at temperatures from about 25° C. to about 75° C., preferably from about 50° C. to about 55° C. under vacuum and at reduced pressure of about 5 mbar to about 20 mbar, for a period of about 10 hours to about 24 hours. Preferably, drying is at about 50-55° C., from about 18 hours and pressure of about 20 mbar.

In another embodiment, the present invention provides a process for the preparation of esomeprazole magnesium, which may be used in the aforementioned process, comprising:
a) contacting esomeprazole or a salt thereof with a magnesium source in the presence of a solvent or a mixture of solvents; and
b) separating the solids by filtration; and
c) concentrating the filtrate to precipitate the crude of esomeprazole magnesium.

Any polymorphic form of esomeprazole free base or salt of esomeprazole known in the prior art including dihydrate form A, dihydrate form B, trihydrate, etc can be used as a starting material. The salt of esomeprazole used herein can be esomeprazole potassium or esomeprazole sodium or esomeprazole magnesium or any other pharmaceutically acceptable salts known in the art. Preferably potassium salt of esomeprazole is being used.

The source of magnesium can be selected from magnesium chloride hexahydrate, magnesium sulphate heptahydrate and the like, preferably magnesium sulphate heptahydrate.

The solvents of a) can be selected from the alcohols such as methanol, ethanol, isopropyl alcohol and the like; halogenated hydrocarbons such as dichloromethane, ethylene dichloride, chloroform and the like or mixtures thereof or their aqueous mixtures in various proportions without limitation. Preferably, methanol and dichloromethane.

The temperature for dissolution can range from about 25° C. to about 35° C., preferably from about 30 to about 35° C.

The time period for dissolution can range from about 30 minutes to about 5 hours, preferably, from about 1 hour to 4 hours.

The time period for complete precipitation of unwanted inorganic solids can be from about 30 minutes to about 5 hours, preferably from about 1 hour to 2 hours.

The unwanted inorganic precipitated solids are separated by filtration.

The precipitation of crude esomeprazole magnesium in c) of the process directly described above, is achieved by, but not limited to evaporation, cooling, drying and the like. Preferably, by evaporation of the solvents from the filtrate to obtain the crude esomeprazole magnesium.

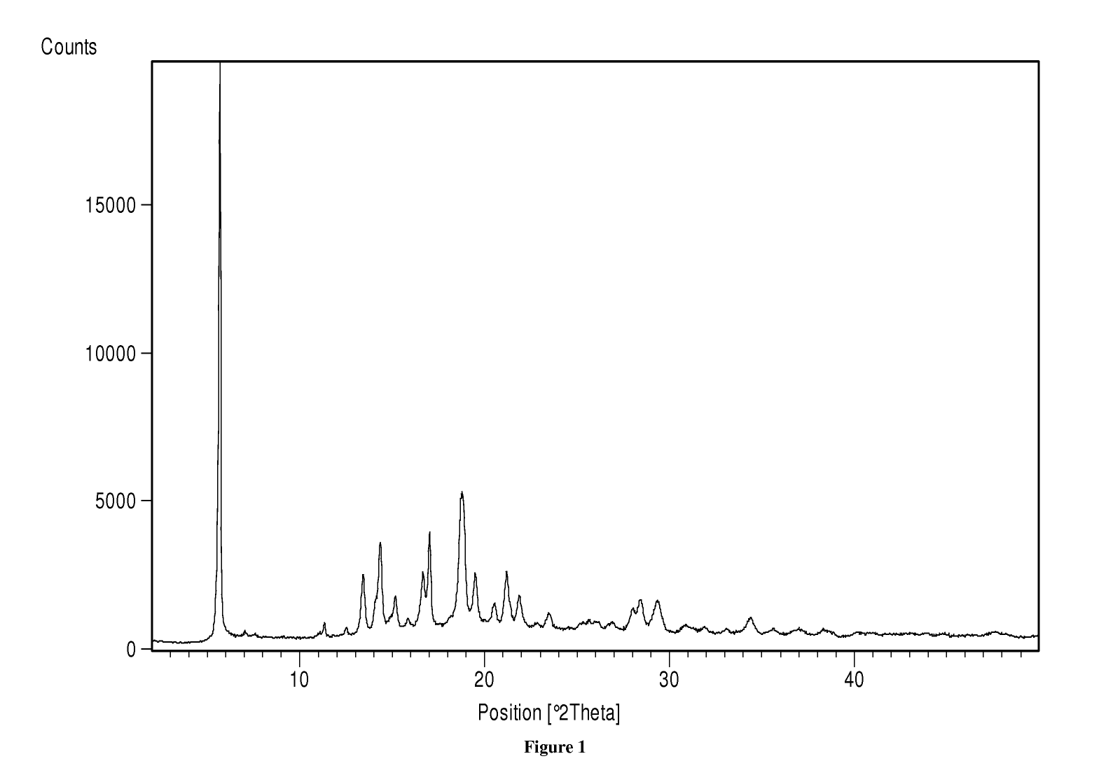

In another embodiment, the present invention provides esomeprazole magnesium dihydrate form A obtained by process herein described, characterized by an X-ray diffractogram, which is substantially in accordance with FIG. 1.

X-ray powder diffraction profiles were obtained using an X-ray Diffractometer (Philips X'Pert Pro, PANalytical. The measurements were carried out with a Pre FIX module programmable divergence slit and anti-scatter Slit (Offset)0.00°; target, Cu; filter, Ni; detector, X'Celerator [1]; Scanning Mode; Active length (2Theta)=2.122°; generator 45 KV; tube current 40 mAmp. The samples were scanned in the full 2θ range of 2-50° with a “time-per-step” optimized to 50 sec. About 300 mg of sample was taken and used to fill the sample holder using Back-loading technique. Then the sample holder was loaded between the X-ray optics-path and scanned using the above-described parameters. Obtained powder X-ray diffraction profiles were integrated using X'Pert High Score Software.

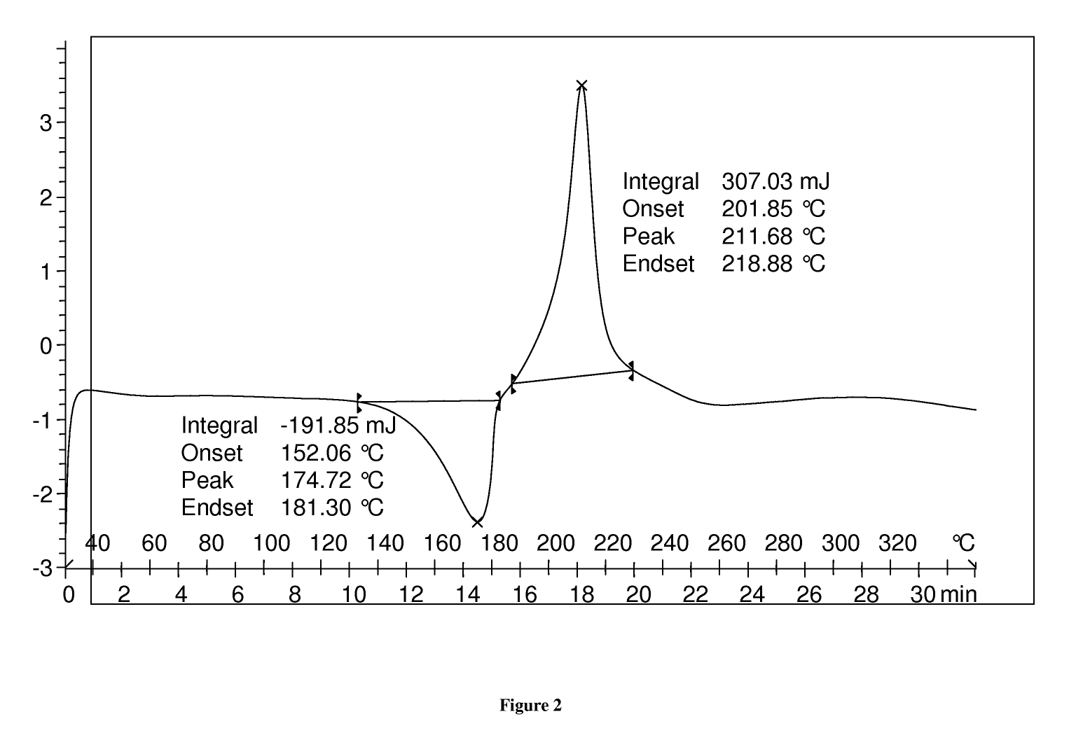

In yet another embodiment, the present invention provides esomeprazole magnesium dihydrate form A obtained by process herein described, characterized by differential scanning calorimetry thermogram, which is substantially in accordance with FIG. 2.

Differential scanning calorimetry (DSC) of esomeprazole Mg dihydrate Form A obtained by the process of present invention is measured by taking approximately 1-2 mg sample was accurately weighed into an Aluminum DSC pan (40 μL) with lid and slightly pierce the lid. The sample was placed into the Mettler Toledo DSC822^{e} equipped with a nitrogen cooling unit and allowed to equilibrate at 30° C. until the stable heat flow reference was seen. A purge nitrogen as dry gas at a flow rate of 50 ml/minute was used to produce inert atmosphere to prevent oxidation of sample during the heating. The sample was then scanned from 30-350° C. at rate of 10° C./minute.

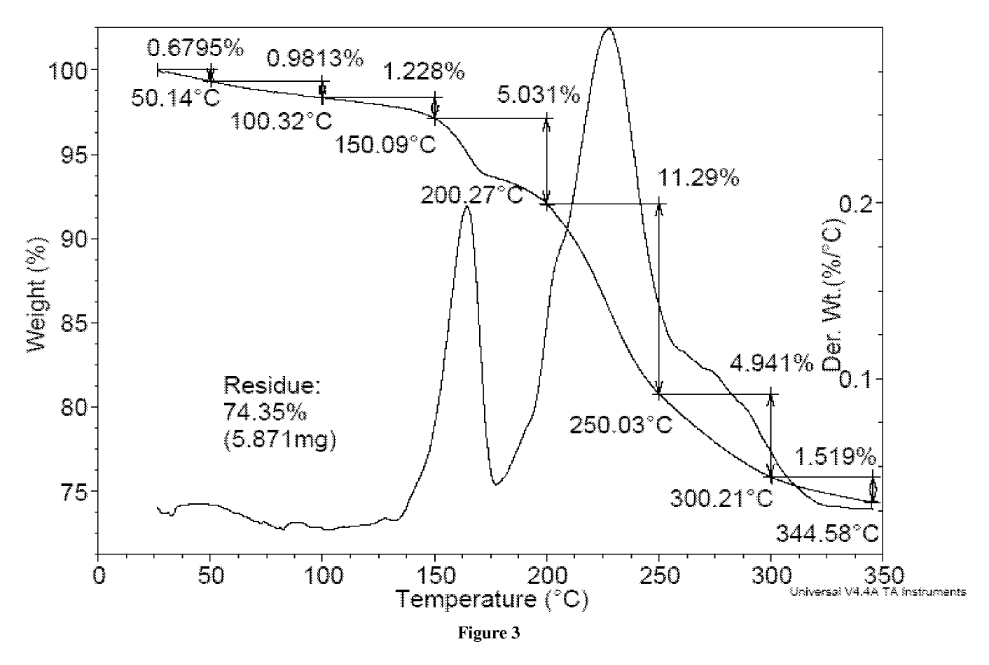

In a still further embodiment, the present invention provides esomeprazole magnesium dihydrate obtained by process herein described, characterized by a thermogravimetric analysis pattern, which is substantially in accordance with FIG. 3.

Thermogravimetric analysis (TGA) of esomeprazole magnesium dihydrate form A obtained by the process of present invention was recorded on TGA Q500 V6.5. Thermogram was recorded at 30° C.-350° C. at the rate of 10° C./min.

In yet another embodiment, the present invention provides esomeprazole magnesium dihydrate form A having a purity of at least about 99.8%, as determined by chiral HPLC.

In a still further embodiment, the present invention provides esomeprazole magnesium dihydrate form A having a purity of at least about 99.9%, as determined by chiral HPLC.

In another embodiment, the present invention provides esomeprazole magnesium dihydrate form A having less than about 0.15% area of (R)-isomer impurity, as determined by chiral HPLC.

In yet another embodiment, the present invention provides esomeprazole magnesium dihydrate form A having less than about 0.1% area of (R)-isomer impurity, as determined by chiral HPLC.

In a still further embodiment, the present invention provides esomeprazole magnesium dihydrate form A having less than about 0.05% area of (R)-isomer impurity as determined by chiral HPLC.

The esomeprazole magnesium dihydrate form A obtained by the process herein described, has the purity greater than 99% by chiral HPLC and any other individual impurity not more than (NMT) 0.1% and total impurities not more than (NMT) 1.0% by chiral HPLC.

In yet another embodiment, esomeprazole magnesium dihydrate form A obtained by the process described herein has a residual organic solvent content of less than the amount recommended for pharmaceutical products, as set forth for example in ICH guidelines and U.S. pharmacopoeia; i.e., less than about 2000 ppm of isopropyl alcohol, less than about 200 ppm of methanol and dichloromethane, less than about 5000 ppm of methyl tertiary butyl ether, dimethyl sulfoxide in the range of about 200 ppm to about 600 ppm and acetic acid, toluene at below the detection limit.

Advantageously, the compound of the present invention, prepared by the process herein described exists in a well defined and stable state, which allows easier characterization and facile handling and storage. Additionally, the compound, prepared by the process herein described, is easier to synthesize in a reproducible manner and thereby easier to handle in a full scale production.

The magnesium salt of esomeprazole dihydrate form A obtained by the process of present invention is substantially free from other forms of magnesium salts of S-omeprazole, such as the corresponding magnesium salt compounds described in prior art, and dihydrates used in the preparation of the dihydrate compound according to the present invention.

The magnesium salt of esomeprazole dihydrate form A obtained by the process of present invention is easily distinguishable from any other crystal form of the magnesium salt of S-omeprazole disclosed in prior art. The compound of the invention is characterized by being highly crystalline, i.e. having a higher crystallinity than any other form of magnesium salt of S-omeprazole disclosed in the prior art.

The degree of crystallinity of magnesium salt of esomeprazole dihydrate form A obtained by the process of present invention can be measured with powder X-ray diffraction (XRD) as described in WO97/4114, which is incorporated herein as reference.

For reducing the % crystallinity, ball milling with stainless steel, ceramic balls, flint or metallic balls can be used.

In an embodiment, the ball milling process is carried out in a ball mill vessel with stainless steel balls rotated at a rate of about 20 rpm to about 100 rpm for about 30 min to about 5 hours at about 25-30° C.

In another embodiment the ball milling process is carried out in a ball mill vessel with 25 mm diameter stainless steel balls numbering about 80 balls to about 100 balls, rotated at a rate of about 30 rpm for about 30 min at 25-30° C.

In an embodiment the present invention provides esomeprazole magnesium dihydrate crystalline form A having a chiral HPLC purity of greater than 99.8 area percent, total impurities of not more than about 1.0 area percent, as determined by HPLC, water content not more than 7.0% and % crystallinity below 70%, preferably between the range of 60% and 70%.

In an embodiment the present invention provides esomeprazole magnesium dihydrate crystalline form A having a chiral HPLC purity of greater than 99.8 area percent, total impurities of not more than about 1.0 area percent, as determined by HPLC, water content not more than 7.0% and % crystallinity below 67%, preferably below 65%.

With the expression “any other form” is meant anhydrates, hydrates, solvates, and polymorphs or amorphous forms thereof disclosed in the prior art. Examples of any other forms of esomeprazole or salt of esomeprazole includes, but are not limited to, anhydrates, monohydrates, dihydrates, sesquihydrates, trihydrates, alcoholates, such as methanolates and ethanolates, and polymorphs or amorphous forms thereof.

In another embodiment, the present invention provides a pharmaceutical composition comprising the magnesium salt of esomeprazole dihydrate form A obtained by the process of present invention, as an active ingredient, in association with a pharmaceutically acceptable carrier, diluent or excipient and optionally other therapeutic ingredients. Useful in the manufacture of a medicament for use in the treatment of a gastric-acid related condition and a method of treating a gastric-acid related condition which method comprises administering to a subject suffering from said condition a therapeutically effective amount of the magnesium salt of esomeprazole dihydrate according to the invention.

The compositions of the invention include compositions suitable for per oral or parental administration. The most preferred route is the oral route. The compositions may be conveniently presented in unit dosage forms, and prepared by any methods known in the art of pharmacy.

In the practice of the invention, the most suitable route of administration as well as the magnitude of a therapeutic dose of the magnesium salt of esomeprazole dihydrate, according to the invention in any given case will depend on the nature and severity of the disease to be treated. The dose, and dose frequency, may also vary according to the age, body weight, and response of the individual patient. Special requirements may be needed for patients having Zollinger-Ellison syndrome, such as a need for higher doses than the average patient. Children and patients with liver diseases generally will benefit from doses that are somewhat lower than the average. Thus, in some conditions it may be necessary to use doses outside the ranges stated below, for example long term treatments may request lower dosage. Such higher and lower doses are within the scope of the present invention. Such daily doses may vary between 5 mg to 300 mg. Dosage forms include capsules, tablets, dispersions, suspensions and the like.

The process for the preparation of esomeprazole magnesium dihydrate form A of the present invention is simple, eco-friendly, robust, reproducible and easily scalable.

The examples which follow will further illustrate the preparation of the compound of the invention. These examples are not intended to limit the scope of the invention as defined hereinabove.

### EXAMPLES

### Example 1

### Preparation of Esomeprazole Magnesium Dihydrate Form a Using Methyl Tertiary Butyl Ether (MTBE) as Solvent

Esomeprazole potassium (100 gm) is suspended in methanol (200 ml) and stirred the solution at 30° C.-35° C. for 10 minutes. Magnesium sulfate heptahydrate (48 g) was added and the reaction mass is stirred for 60 min at 30° C.-35° C. Methylene chloride (400 ml) was added and the contents were further stirred for 1 h at 30° C.-35° C. The reaction mass is filtered and filtrate is concentrated and cooled to 25° C. to 30° C. A mixture of methyl tertiary butyl ether and water (400 ml) was added and stirred for 4 to 5 hours and the precipitated esomeprazole magnesium dihydrate having form A is isolated by filtration under nitrogen atmosphere washed with methyl tertiary butyl ether (300 ml) and dried under vacuum at 50-55° C. to yield (35 gm, 38%)

### Purity by Chiral HPLC: 99.82% ee.

### Example 2

### Preparation of Esomeprazole Magnesium Dihydrate Form a Using Mixture of Methyl Tertiary Butyl Ether (MTBE)/Isopropyl Alcohol (IPA) and Water) as Solvents

Potassium salt of esomeprazole (200 gm) is suspended in methanol (400 ml) and stirred the solution at 30-35° C. for 10 minutes. Magnesium sulfate heptahydrate (96 g) was added at 30° C.-35° C. and reaction mass is stirred for 1 h at 30° C.-35° C. Methylene chloride (800 ml) was added and the contents were further stirred for 1 h at 30° C.-35° C. The reaction mass is filtered and filtrate is concentrated and cooled to 25° C. to 30° C. A mixture of methyl tertiary-butyl ether (360 ml), isopropyl alcohol (360 ml) and water (80 ml) was added and stirred for 4 to 5 hours and the precipitated esomeprazole magnesium dihydrate having form A is isolated by filtration under nitrogen atmosphere washed with isopropyl alcohol (400 ml) and dried under vacuum at 50° C.-55° C. to yield (67 gm, 37%)

### Purity by Chiral HPLC: 99.98%.

Organic volatile impurities (O.V.I):
Isopropyl alcohol: 3167 ppm.
Methanol: Below detection limit.
Methylene chloride: Below detection limit
Methyl tertiary butyl ether: δ 1 ppm.
Toluene: Below detection limit.

### Example 3

### Preparation of Esomeprazole Magnesium Dihydrate

Esomeprazole Magnesium dihydrate (150 g, degree of crystallinity 72%) is taken in a ball mill vessel (6 L capacity) with stainless steel balls (25 mm diameter, 80 balls). The ball mill vessel is rotated at a rate of 30 rpm for 30 min at 25-30° C. Thereafter, the material is unloaded to yield esomeprazole magnesium dihydrate with the degree of crystallinity of 63.8%.

### Yield: 142 gm.

The degree of crystallinity of the obtained product can be measured with powder X-ray diffraction (XRD) as described in WO97/4114, herein incorporated as reference.

We claim:

1. Esomeprazole magnesium dihydrate crystalline form A having a chiral HPLC purity greater than 99.8 area percent, total impurities of not more than about 1.0 area percent, as determined by HPLC and water content of not more than 7.0%.

2. Esomeprazole magnesium dihydrate crystalline form A having a chiral HPLC purity of greater than 99.8 area percent, total impurities of not more than about 1.0 area percent, as determined by HPLC, water content of not more than 7.0% and % crystallinity below 70%.

3. The compound of claim 1, characterized by an X-ray diffractogram, which is substantially in accordance with FIG. 1.

4. The compound of claim 1, characterized by a differential scanning calorimetry thermogram, which is substantially in accordance with FIG. 2.

5. The compound of claim 1, characterized by a thermogravimetric analysis pattern, which is substantially in accordance with FIG. 3.

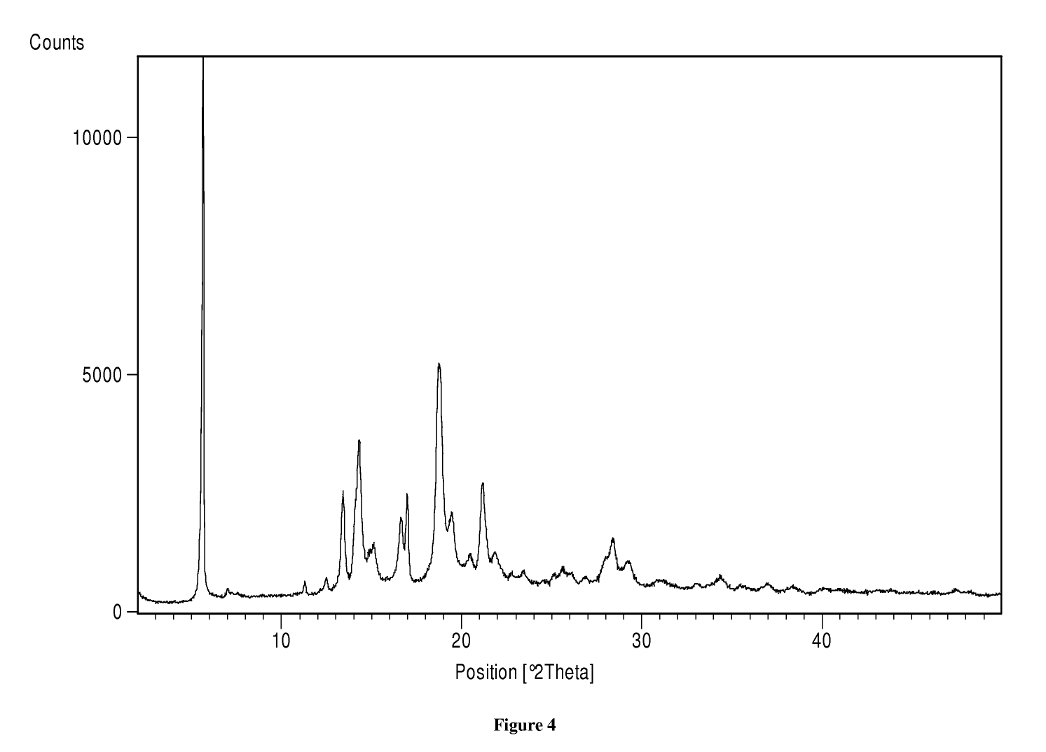

6. The compound of claim 2, characterized by an X-ray diffractogram, which is substantially in accordance with FIG. 4.

7. (canceled)

8. The process of claim 15, wherein the recovery comprises filtration followed by washing with isopropyl alcohol.

9. The process of claim 15, wherein the recovered esomeprazole magnesium dihydrate form A is further dried at temperatures from about 30° C. to about 55° C. under vacuum for a period of about 1 hour to about 15 hours.

10. The process of claim 15, wherein the resultant crystalline dihydrate of esomeprazole magnesium is subjected to ball milling.

11. The process of claim 15, wherein the esomeprazole magnesium is prepared by a process comprising:
a) contacting esomeprazole or a salt thereof with a magnesium source in the presence of solvent or a mixture of solvents;
b) separating the solids by filtration; and
c) concentrating the filtrate to obtain the crude of esomeprazole magnesium.

12. The process of claim 11, wherein the esomeprazole salt is esomeprazole potassium.

13. The process of claim 11, wherein the magnesium source is magnesium heptahydrate.

14. The process of claim 11, wherein the organic solvents are methanol and dichloromethane.

15. A process for preparation of esomeprazole magnesium dihydrate, as in any of claim 1 or 2, comprising:
a) providing a suspension of esomeprazole magnesium by contacting it with isopropyl alcohol and methyl tertiary butyl ether in combination with water
b) precipitating the solid by stifling at about 30° C.; and
c) recovering the solid to obtain the crystalline dihydrate form A of esomeprazole magnesium.
